(19) United States
(12) Patent Application Publication
(10) Pub. No.: US 2026/0017257 A1
(43) Pub. Date: Jan. 15, 2026

(54) System and Method for Automated Prompt Tuning for Generative Artificial Intelligence (AI) Model-generated Structured Documents

(71) Applicant: Onpoint Healthcare Partners, Inc., Irving, TX (US)

(72) Inventors: Rodney E. Haynes, Tarpon Springs, FL (US); James W. Boswell, Flower Mound, TX (US); Jason Cochran, Jonesboro, AR (US)

(21) Appl. No.: 19/268,409

(22) Filed: Jul. 14, 2025

Related U.S. Application Data
(60) Provisional application No. 63/670,579, filed on Jul. 12, 2024.

(51) Int. Cl. G06F 16/2453; G06F 40/205; G16H 10/60

(52) U.S. Cl. CPC G06F 16/2453; G06F 40/205; G16H 10/60

(57) ABSTRACT

A method, computer program product, and computing system for processing an intermediary structured document generated by a first generative artificial intelligence (AI) model using a plurality of predefined prompts. The intermediary structured document is compared with a curated structured document. A scoring of the intermediary structured document is generated based upon, at least in part, the comparing of the intermediary structured document with a curated structured document. One or more revisions for the plurality of predefined prompts are generated by processing the scoring of the intermediary structured document using a second generative AI model.

### PRIORITY APPLICATION

This application claims the benefit of U.S. Provisional Application No. 63/670,579, filed on 12 Jul. 2024, the entire contents of which are herein incorporated by reference.

### BACKGROUND

Generative artificial intelligence (AI) models, particularly large language models, are increasingly being used to automate the creation of structured documents such as medical notes. These systems typically generate documents by responding to user prompts that may include specific data fields or contextual information, such as patient demographics, symptoms, and diagnoses. The flexibility of these models allows them to adapt to a wide range of document templates and styles, especially when provided with clear and detailed prompts. Some implementations even integrate with electronic health records to pull structured data directly into the prompt, which can enhance both accuracy and relevance. Prompts can be customized to ensure the inclusion of specific sections, terminology, or compliance requirements.

However, several limitations persist, particularly when prompts are not carefully optimized. Vague or underspecified prompts can result in outputs that are inaccurate or incomplete, with missing sections, irrelevant content, or hallucinated details. If the prompt does not clearly map user data to the required document structure, the generative AI model may misplace or omit critical information. This lack of optimization can also lead to inconsistency in document formats, making downstream processing or review more difficult, and may cause the generative AI model to deviate from required templates over time. These issues introduce clinical and legal risks, as inaccurate notes can have significant consequences and expose organizations to liability. Additionally, poorly structured prompts can make it difficult to trace how specific data points were incorporated, complicating audits or legal reviews.

The burden of prompt engineering often falls on users, who may need to learn specialized techniques or engage in iterative refinement to achieve reliable outputs, increasing time and effort. To mitigate these challenges, conventional approaches include using standardized, pre-validated prompt templates for common document types, feeding structured data directly into prompts to minimize ambiguity, implementing human-in-the-loop review or automated validation to catch errors, and regularly reviewing and refining prompts based on output quality and user feedback.

### SUMMARY OF DISCLOSURE

In one example implementation, a computer-implemented method executed on a computing device may include, but is not limited to, processing an intermediary structured document generated by a first generative artificial intelligence (AI) model using a plurality of predefined prompts. The intermediary structured document is compared with a curated structured document. A scoring of the intermediary structured document is generated based upon, at least in part, the comparing of the intermediary structured document with a curated structured document. One or more revisions for the plurality of predefined prompts are generated by processing the scoring of the intermediary structured document using a second generative AI model.

One or more of the following example features may be included. The intermediary structured document may be a medical record generated by the first generative AI model using the plurality of predefined prompts and medical data. The curated structured document may be a medical record annotated by a medical professional. Comparing the intermediary structured document with a curated structured document may include parsing the intermediary structured document into a plurality of sections and the curated structured document into a plurality of sections. Comparing the intermediary structured document with a curated structured document may include comparing each section of the plurality of sections from the intermediary structured document with each corresponding section of the plurality of sections from the curated structured document. Generating the scoring of the intermediary structured document may include generating a weighted score for each section of the plurality of sections from the intermediary structured document using a plurality of weights. Generating the one or more revisions for the plurality of predefined prompts may include generating a plurality of revisions to be applied incrementally to the plurality of predefined prompts over a plurality of updates of the plurality of predefined prompts using a predefined relative prioritization.

In another example implementation, a computer program product resides on a computer readable medium that has a plurality of instructions stored on it. When executed by a processor, the instructions cause the processor to perform operations that may include, but are not limited to, processing an intermediary structured document generated by a first generative artificial intelligence (AI) model using a plurality of predefined prompts. The intermediary structured document is compared with a curated structured document. A scoring of the intermediary structured document is generated based upon, at least in part, the comparing of the intermediary structured document with a curated structured document. One or more revisions for the plurality of predefined prompts are generated by processing the scoring of the intermediary structured document using a second generative AI model.

One or more of the following example features may be included. The intermediary structured document may be a medical record generated by the first generative AI model using the plurality of predefined prompts and medical data. The curated structured document may be a medical record annotated by a medical professional. Comparing the intermediary structured document with a curated structured document may include parsing the intermediary structured document into a plurality of sections and the curated structured document into a plurality of sections. Comparing the intermediary structured document with a curated structured document may include comparing each section of the plurality of sections from the intermediary structured document with each corresponding section of the plurality of sections from the curated structured document. Generating the scoring of the intermediary structured document may include generating a weighted score for each section of the plurality of sections from the intermediary structured document using a plurality of weights. Generating the one or more revisions for the plurality of predefined prompts may include generating a plurality of revisions to be applied incrementally to the plurality of predefined prompts over a plurality of updates of the plurality of predefined prompts using a predefined relative prioritization.

In another example implementation, a computing system includes at least one processor and at least one memory architecture coupled with the at least one processor, wherein the at least one processor is configured to process an intermediary structured document generated by a first generative artificial intelligence (AI) model using a plurality of predefined prompts. The intermediary structured document is compared with a curated structured document. A scoring of the intermediary structured document is generated based upon, at least in part, the comparing of the intermediary structured document with a curated structured document. One or more revisions for the plurality of predefined prompts are generated by processing the scoring of the intermediary structured document using a second generative AI model.

One or more of the following example features may be included. The intermediary structured document may be a medical record generated by the first generative AI model using the plurality of predefined prompts and medical data. The curated structured document may be a medical record annotated by a medical professional. Comparing the intermediary structured document with a curated structured document may include parsing the intermediary structured document into a plurality of sections and the curated structured document into a plurality of sections. Comparing the intermediary structured document with a curated structured document may include comparing each section of the plurality of sections from the intermediary structured document with each corresponding section of the plurality of sections from the curated structured document. Generating the scoring of the intermediary structured document may include generating a weighted score for each section of the plurality of sections from the intermediary structured document using a plurality of weights. Generating the one or more revisions for the plurality of predefined prompts may include generating a plurality of revisions to be applied incrementally to the plurality of predefined prompts over a plurality of updates of the plurality of predefined prompts using a predefined relative prioritization.

The details of one or more example implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

### BRIEF DESCRIPTION OF THE DRAWINGS

FIG. 1 is an example diagrammatic view of a storage system and an automated prompt tuning process coupled to a distributed computing network according to one or more example implementations of the disclosure;

FIG. 2 is an example flowchart of automated prompt tuning process according to one or more example implementations of the disclosure; and

FIG. 3 is an example diagrammatic view of an automated prompt tuning process according to one or more example implementations of the disclosure.

Like reference symbols in the various drawings indicate like elements.

### DETAILED DESCRIPTION

### System Overview

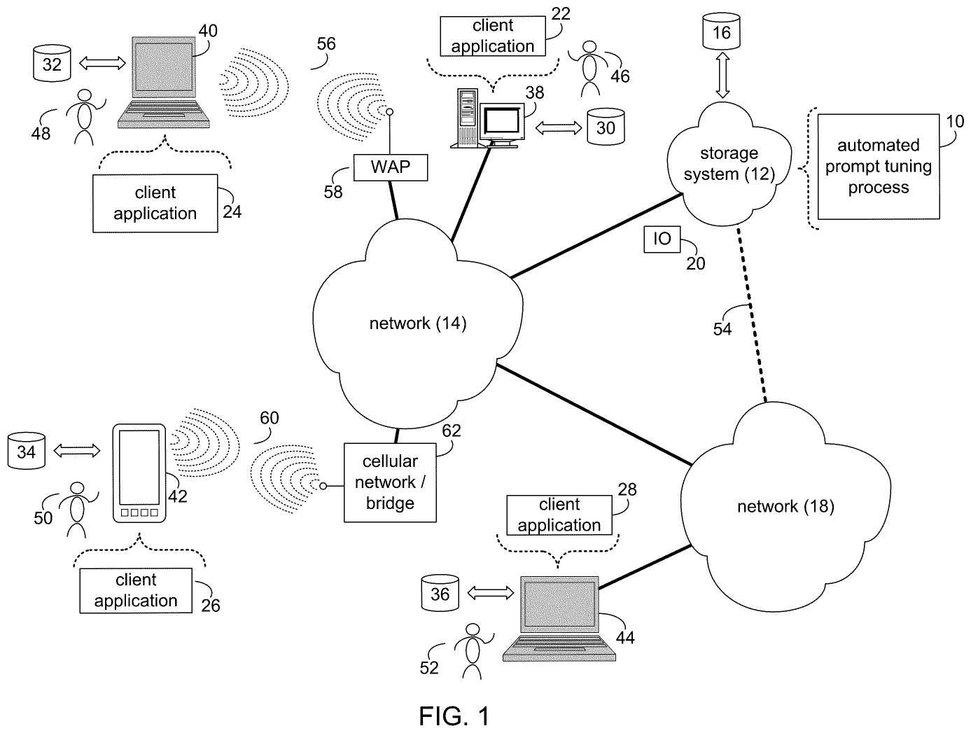

Referring to FIG. 1, there is shown automated prompt tuning process 10 that may reside on and may be executed by storage system 12, which may be connected to network 14 (e.g., the Internet or a local area network). Examples of storage system 12 may include, but are not limited to: a Network Attached Storage (NAS) system, a Storage Area Network (SAN), a personal computer with a memory system, a server computer with a memory system, and a cloud-based device with a memory system.

As is known in the art, a SAN may include one or more of a personal computer, a server computer, a series of server computers, a minicomputer, a mainframe computer, a RAID device, and a NAS system. The various components of storage system 12 may execute one or more operating systems, examples of which may include but are not limited to: Microsoft® Windows®; Mac® OS X®; Red Hat® Linux®, Windows® Mobile, Chrome OS, Blackberry OS, Fire OS, or a custom operating system. (Microsoft and Windows are registered trademarks of Microsoft Corporation in the United States, other countries, or both; Mac and OS X are registered trademarks of Apple Inc. in the United States, other countries or both; Red Hat is a registered trademark of Red Hat Corporation in the United States, other countries or both; and Linux is a registered trademark of Linus Torvalds in the United States, other countries or both).

The instruction sets and subroutines of automated prompt tuning process 10, which may be stored on storage device 16 included within storage system 12, may be executed by one or more processors (not shown) and one or more memory architectures (not shown) included within storage system 12. Storage device 16 may include but is not limited to: a hard disk drive; a tape drive; an optical drive; a RAID device; a random-access memory (RAM); a read-only memory (ROM); and all forms of flash memory storage devices. Additionally/alternatively, some portions of the instruction sets and subroutines of automated prompt tuning process 10 may be stored on storage devices (and/or executed by processors and memory architectures) that are external to storage system 12.

Network 14 may be connected to one or more secondary networks (e.g., network 18), examples of which may include but are not limited to: a local area network; a wide area network; or an intranet, for example.

Various IO requests (e.g., IO request 20) may be sent from client applications 22, 24, 26, 28 to storage system 12. Examples of IO request 20 may include but are not limited to data write requests (e.g., a request that content be written to storage system 12) and data read requests (e.g., a request that content be read from storage system 12).

The instruction sets and subroutines of client applications 22, 24, 26, 28, which may be stored on storage devices 30, 32, 34, 36 (respectively) coupled to client electronic devices 38, 40, 42, 44 (respectively), may be executed by one or more processors (not shown) and one or more memory architectures (not shown) incorporated into client electronic devices 38, 40, 42, 44 (respectively). Storage devices 30, 32, 34, 36 may include but are not limited to: hard disk drives; tape drives; optical drives; RAID devices; random access memories (RAM); read-only memories (ROM), and all forms of flash memory storage devices. Examples of client electronic devices 38, 40, 42, 44 may include, but are not limited to, personal computer 38, laptop computer 40, smartphone 42, notebook computer 44, a server (not shown), a data-enabled, cellular telephone (not shown), and a dedicated network device (not shown).

Users 46, 48, 50, 52 may access storage system 12 directly through network 14 or through secondary network 18. Further, storage system 12 may be connected to network 14 through secondary network 18, as illustrated with link line 54.

The various client electronic devices may be directly or indirectly coupled to network 14 (or network 18). For example, personal computer 38 is shown directly coupled to network 14 via a hardwired network connection. Further, notebook computer 44 is shown directly coupled to network 18 via a hardwired network connection. Laptop computer 40 is shown wirelessly coupled to network 14 via wireless communication channel 56 established between laptop computer 40 and wireless access point (e.g., WAP) 58, which is shown directly coupled to network 14. WAP 58 may be, for example, an IEEE 802.11a, 802.11b, 802.11g, 802.11n, Wi-Fi, and/or Bluetooth device that is capable of establishing wireless communication channel 56 between laptop computer 40 and WAP 58. Smartphone 42 is shown wirelessly coupled to network 14 via wireless communication channel 60 established between smartphone 42 and cellular network/bridge 62, which is shown directly coupled to network 14.

Client electronic devices 38, 40, 42, 44 may each execute an operating system, examples of which may include but are not limited to Microsoft® Windows®; Mac® OS X®; Red Hat® Linux®, Windows® Mobile, Chrome OS, Blackberry OS, Fire OS, or a custom operating system. (Microsoft and Windows are registered trademarks of Microsoft Corporation in the United States, other countries, or both; Mac and OS X are registered trademarks of Apple Inc. in the United States, other countries or both; Red Hat is a registered trademark of Red Hat Corporation in the United States, other countries or both; and Linux is a registered trademark of Linus Torvalds in the United States, other countries or both).

In some implementations, as will be discussed below in greater detail, an automated prompt tuning process, such as automated prompt tuning process 10 of FIG. 1, may include but is not limited to, processing an intermediary structured document generated by a first generative artificial intelligence (AI) model using a plurality of predefined prompts. The intermediary structured document is compared with a curated structured document. A scoring of the intermediary structured document is generated based upon, at least in part, the comparing of the intermediary structured document with a curated structured document. One or more revisions for the plurality of predefined prompts are generated by processing the scoring of the intermediary structured document using a second generative AI model.

For example purposes only, storage system 12 will be described as being a network-based storage system that includes a plurality of electro-mechanical backend storage devices. However, this is for example purposes only and is not intended to be a limitation of this disclosure, as other configurations are possible and are considered to be within the scope of this disclosure.

### The Automated Prompt Tuning Process

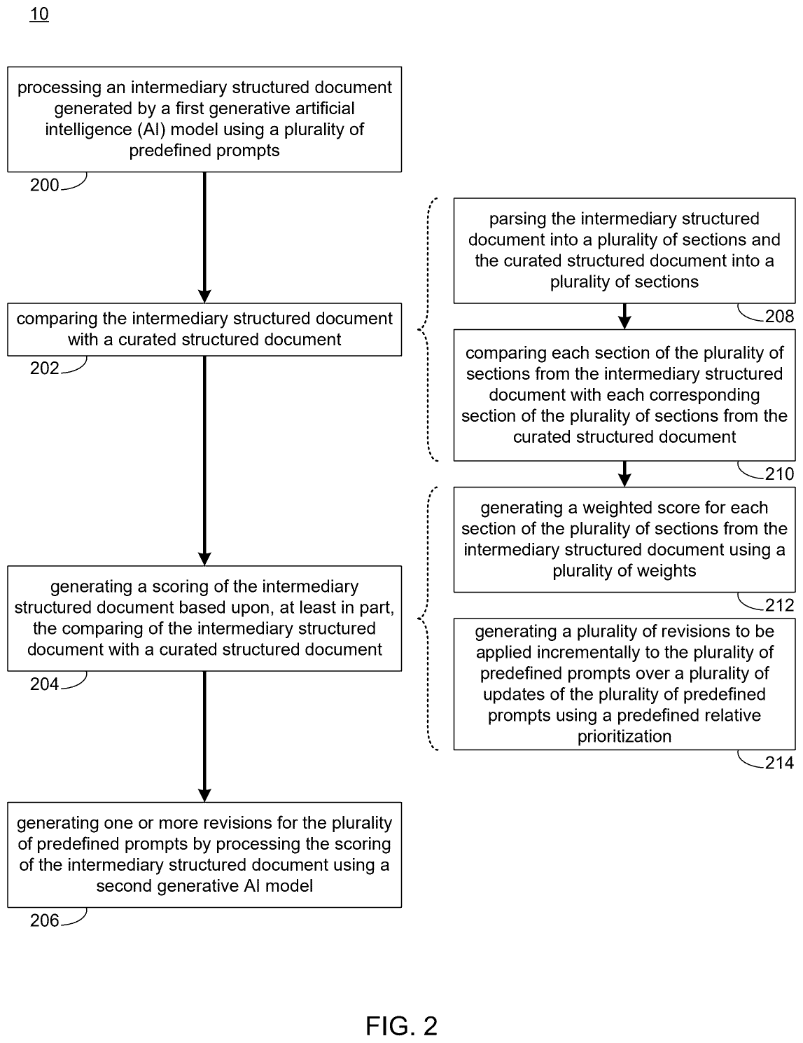

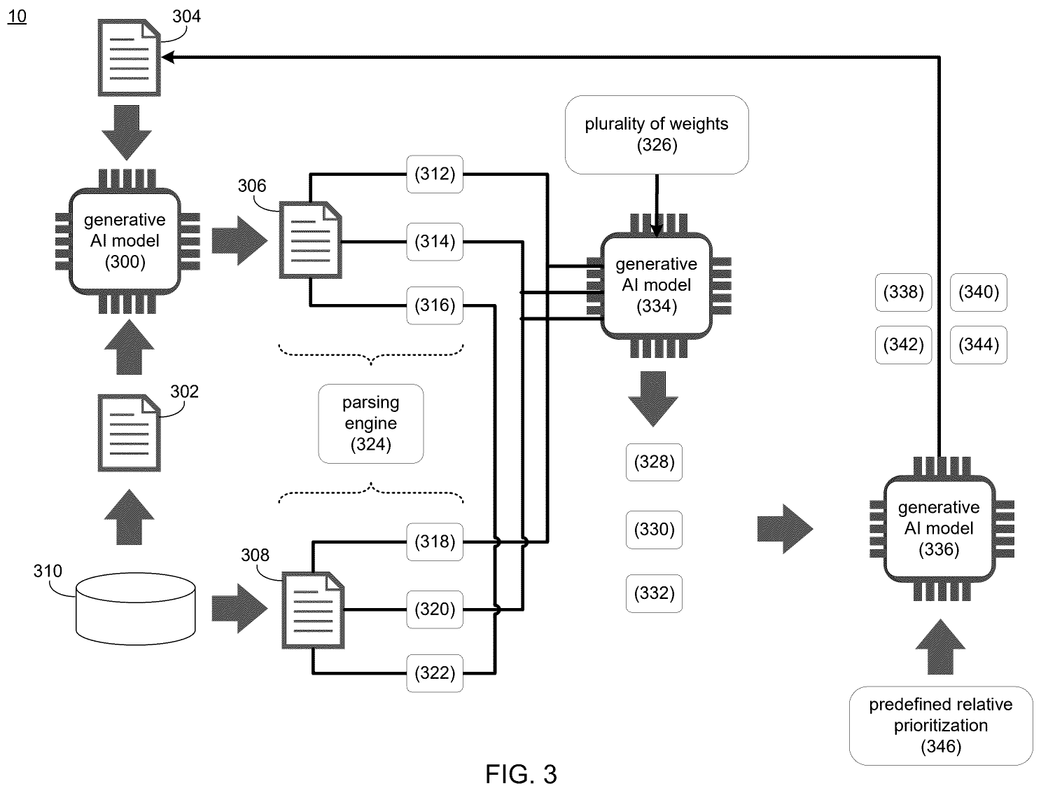

Referring also to the examples of FIGS. 2-3 and in some implementations, automated prompt tuning process 10 may process 200 an intermediary structured document generated by a first generative artificial intelligence (AI) model using a plurality of predefined prompts. The intermediary structured document is compared 202 with a curated structured document. A scoring of the intermediary structured document is generated 204 based upon, at least in part, the comparing of the intermediary structured document with a curated structured document. One or more revisions for the plurality of predefined prompts are generated 206 by processing the scoring of the intermediary structured document using a second generative AI model.

As will be discussed in greater detail below, implementations of the present disclosure may allow for curated or validated examples of structured documents to iteratively refine prompts, reducing errors, and improving quality over time. For example, AI-generated structured documents often contain errors (e.g., misplaced information in sections like HPI or Review of Systems) and are vulnerable to subjectivity in validation. Further, generative AI models conventionally do not adapt to provider feedback or evolving standards. As will be discussed in greater detail below, automated prompt tuning process 10 automatically tags curated structured documents to identify changes and corrections using objective measurements. Using this feedback, automated prompt tuning process 10 generates new prompts and/or revisions to prompts on a section-by-section basis.

In some implementations, automated prompt tuning process 10 processes 200 an intermediary structured document generated by a first generative artificial intelligence (AI) model using a plurality of predefined prompts. For example, structured documents are organized according to predefined templates or schemas, which ensure that information is consistently captured, easily interpreted, and suitable for downstream processing or compliance. In a healthcare example, medical notes such as progress notes, discharge summaries, operative reports, and consultation notes are typically structured into standardized sections. A common format is the SOAP note, which includes Subjective (patient's reported symptoms and history), Objective (clinician's observations, vital signs, and test results), Assessment (diagnosis or clinical impression), and Plan (treatment plan, follow-up, and recommendations). These notes can be generated manually by clinicians entering data into electronic health record templates, or increasingly, by generative AI models that synthesize both structured and unstructured data—such as patient demographics, lab results, and dictated summaries—into the required format based on prompts and user input.

In another example, legal structured documents like contracts, pleadings, discovery requests, and compliance checklists follow strict templates with sections such as recitals, definitions, operative clauses, representations and warranties, covenants, and signature blocks. These are often created using document automation tools or AI models that populate templates with client-specific data, legal clauses, and jurisdictional requirements based on user prompts or questionnaire responses.

In another example, financial structured documents include balance sheets, income statements, audit reports, and regulatory filings, all organized according to accounting standards with sections for assets, liabilities, equity, revenues, expenses, and explanatory notes. Data for these documents is typically pulled from accounting systems or spreadsheets and formatted into standardized reports, sometimes with narrative sections generated by AI to summarize key trends or compliance issues.

In yet another example concerning business operations generally, structured documents such as project status reports, risk assessments, meeting minutes, and incident reports are common. These documents usually include fields for project identifiers, objectives, milestones, risks, action items, and responsible parties. Generation methods may vary: users may fill out predefined templates manually, may use template-based automation to merge data from databases or forms, or may rely on AI-driven tools that prompt users for required data and assemble the information into a consistent report format. In all these cases, the organization or structure of the document may be dictated by the template or schema, and generation can be manual, automated, or AI-driven, with increasing reliance on AI for synthesizing and structuring complex data into standardized formats.

In some implementations, the intermediary structured document is a medical record generated by the first generative AI model using the plurality of predefined prompts and medical data. For example, generative AI models are a class of artificial intelligence systems designed to create new content, such as text, images, audio, or code, based on patterns learned from large datasets. Unlike traditional AI models that focus on classification or prediction, generative models produce original outputs that resemble the data they were trained on. The most prominent generative AI models in recent years are large language models (LLMs), such as those based on the transformer architecture. These models are trained on vast corpora of text and can generate coherent and contextually relevant language in response to prompts. They are capable of tasks such as drafting documents, answering questions, summarizing information, translating languages, and even creating poetry or stories.

Generative AI models are not limited to text. For example, in the visual domain, generative AI models like generative adversarial networks (GANs) and diffusion models can create realistic images. In audio, generative models can synthesize speech, music, or sound effects.

The core mechanism behind generative AI models involves learning the statistical relationships and structures within the training data. When given a prompt or input, the generative AI model predicts the most likely next element (word, pixel, note, etc.) and continues this process iteratively to produce a complete output. The quality and relevance of the generated content depend on the size and diversity of the training data, the architecture of the model, and the specificity of the input prompt.

Referring also to FIG. 3, a first generative AI model (e.g., first generative AI model 300) processes input data (e.g., input data 302) and a plurality of predefined prompts (e.g., plurality of predefined prompts 304) to generate an intermediary structured document (e.g., intermediary structured document 306). In one example, intermediary structured document 306 is a medical record generated by first generative AI model 300 by processing input medical data (e.g., input data 302) and the plurality of prompts (e.g., plurality of prompts 304). In this example, automated prompt tuning process 10 extracts relevant portions of medical data for a particular section of intermediary structured document 306 from input medical data 302 to automatically populate intermediary structured document 306 according to the constraints and provisions of plurality of prompts 304. While an example of a medical record has been provided for intermediary structured document 306, it will be appreciated that any type of structured document may be generated by first generative AI model 300 within the scope of the present disclosure.

In some implementations, automated prompt tuning process 10 compares 202 the intermediary structured document with a curated structured document. For example, a curated structured document is an exemplary version of the structured document that is corrected and/or annotated by a user or another generative AI model. In some implementations, when processing 200 intermediary structured document 306, automated prompt tuning process 10 may determine a type or domain for intermediary structured document 306 and retrieves the curated structured document (e.g., curated structured document 308) from a database of curated structured documents (e.g., database 310). In one example where intermediary structured document 306 is a medical record generated by first generative AI model 300, curated structured document 308 is a medical record annotated by a medical professional. In this example, automated prompt tuning process 10 determines that intermediary structured document 306 is a medical record and retrieves a corresponding curated structured document 308. In some implementations, curated structured document 308 is a consistent control. For instance, when processing intermediary structured document 306, automated prompt tuning process 10 may retrieve a consistent control version of intermediary structured document 306. In this example, curated structured document 308 is a vetted to be an unbiased and grounded version of intermediary structured document 306. In another example, curated structured document 308 is a randomly selected version of a corresponding intermediary structured document retrieved from database 310. In this example, curated structured document 308 is selected from a plurality of structured documents created within a threshold period of time (e.g., within the last two weeks). As these randomly selected and recently curated structured documents are not as thoroughly vetted as a consistent control, the curated structured documents may potentially include bias or preferences of the user. In another example, curated structured document 308 may include content extracted from other documents or portions of a structured document. In this example, curated structured document 308 may be generated by automated prompt tuning process 10 using content from verified or vetted sources within database 310.

In some implementations, comparing 202 the intermediary structured document with a curated structured document includes determining a similarity between intermediary structured document 306 and curated structured document 308. For example and as will be described in greater detail below, similarity may be defined as a metric or count of common content and/or differences between intermediary structured document 306 and curated structured document 308.

In some implementations, comparing 202 the intermediary structured document with a curated structured document includes parsing 208 the intermediary structured document into a plurality of sections and the curated structured document into a plurality of sections. For example, intermediary structured document 306 and corresponding curated structured document 308 may each include predefined sections where content of particular types and forms may be recorded by users and/or generative AI models when accessing the structured document. In one example where intermediary structured document 306 is a medical record, automated prompt tuning process 10 parses 208 intermediary structured document 306 into a plurality of sections (e.g., plurality of sections 312, 314, 316) and curated structured document 308 into a plurality of sections (e.g., plurality of sections 318, 320, 322). In this example, sections 312, 314, 316 may concern patient identification, chief complaint (CC), history of present illness (HPI), past medical history (PMH), medications, allergies, review of systems (ROS), assessment, and other medical related information obtained during a medical encounter. Accordingly, automated prompt tuning process 10 parses 208 intermediary structured document 306 and curated structured document 308 using a parsing engine (e.g., parsing engine 324) that is configured to parse text from intermediary structured document 306 and curated structured document 308 on a section-per-section basis. In this manner, automated prompt tuning process 10 organizes content from intermediary structured document 306 and curated structured document 308 into their respective sections.

In some implementations, comparing 202 the intermediary structured document with a curated structured document includes comparing 210 each section of the plurality of sections from the intermediary structured document with each corresponding section of the plurality of sections from the curated structured document. For example and referring again to FIG. 3, automated prompt tuning process 10 compares each section of plurality of sections 312, 314, 316 from intermediary structured document 306 with each corresponding section of plurality of sections 318, 320, 322 from curated structured document 308. In this example, automated prompt tuning process 10 compares 210 section 312 from intermediary structured document 306 to corresponding section 318 from curated structured document 308; section 314 from intermediary structured document 306 to corresponding section 320 from curated structured document 308; and section 316 from intermediary structured document 306 to corresponding section 322 from curated structured document 308.

In one example, suppose that section 312 from intermediary structured document 306 includes the following text: “1. VITALS: 2. CONSTITUTIONAL: 3. HEENT: 4. Neck: 5. CARDIOVASCULAR: Negative for chest pain and palpitations. 6. PULMONARY: Negative . . . ” and suppose that corresponding section 318 from curated structured document 308 includes the following text: “1. VITALS: 2. CONSTITUTIONAL: Positive for weight gain. No fevers, chills. 3. HEENT: Positive for snoring. No dysphagia . . . ”. In this example, automated prompt tuning process 10 compares 210 section 312 from intermediary structured document 306 to corresponding section 318 from curated structured document 308 for errors in a textual output as follows: “1. Original Note: ‘VITALS:’ Provider Note: ‘VITALS:’ Error Category: Same Meaning Error Impact: No Difference 2. Original Note: ‘CONSTITUTIONAL:’ Provider Note: ‘CONSTITUTIONAL: Positive for weight gain. No fevers, chills’ Error Category: Missing Content Error Impact: . . . ”. From this example, automated prompt tuning process 10 compares 210 section 312 from intermediary structured document 306 to corresponding section 318 from curated structured document 308 to identify objective error types in intermediary structured document 306. Examples of error types include identifying added content, identifying missing content, identifying inaccurate representations of content, etc. In this manner, automated prompt tuning process 10 is able to automatically identify differences between intermediary structured document 306 and curated structured document 308 on a section-by-section basis.

In some implementations, automated prompt tuning process 10 generates 204 a scoring of the intermediary structured document based upon, at least in part, the comparing of the intermediary structured document with a curated structured document. A scoring is a numerical representation of the comparison of intermediary structured document 306 to curated structured document 308. In one example, the scoring indicates a percentage of matching content between intermediary structured document 306 and curated structured document 308 (i.e., a higher score indicates a greater match between content of intermediary structured document 306 and content of curated structured document 308). In another example, the scoring indicates a percentage of unique content between intermediary structured document 306 and curated structured document 308 (i.e., a higher score indicates a greater difference between content of intermediary structured document 306 and content of curated structured document 308). In another example, the scoring indicates a number of error types from a plurality of predefined error types.

In some implementations, generating 204 the scoring of the intermediary structured document includes generating 212 a weighted score for each section of the plurality of sections from the intermediary structured document using a plurality of weights. For example and as described above, automated prompt tuning process 10 may generate 204 a scoring using the error types from a plurality of predefined error types. In some implementations, each error type may have varying impact on the scoring and accuracy of intermediary structured document 306 relative to curated structured document 308. Accordingly, automated prompt tuning process 10 may generate 212 a weighted score for each section of the plurality of sections where the weighting of each score is defined using a plurality of weights (e.g., plurality of weights 326). In one example, plurality of weights 326 includes a weighting of each error type, such that certain error types are weighted more or less than other error types (e.g., a weight of “10” for section misplacement and a weight of “5” for formatting issues). In some implementations, the weighting of plurality of weights 326 is user-defined and/or defined by automated prompt tuning process 10. In some implementations, automated prompt tuning process 10 generates 212 a weighted score for each error type (e.g., weighted scores 328, 330, 332) using a generative AI model (e.g., generative AI model 334) by processing each section of intermediary structured document 306, each corresponding section of curated structured document 308, and plurality of weights 326.

In some implementations, automated prompt tuning process 10 generates 206 one or more revisions for the plurality of predefined prompts by processing the scoring of intermediary structured document using a second generative AI model. For example, a revision for the plurality of predefined prompts is a correction to a predefined prompt, an addition to a predefined prompt, an addition of a new prompt, and/or the removal of a predefined prompt from the plurality of predefined prompts. In some implementations, automated prompt tuning process 10 provides the scoring of intermediary structured document 306 to second generative AI model 336 to generate the one or more revisions (e.g., revisions 338, 340, 342, 344). Examples of revisions 338, 340, 342, 344 include textual directions provided to first generative AI model 300 for particular predefined prompts. In one example, revisions 338, 340, 342, 344 include directions concerning particular sections of intermediary structured document 306 (e.g., “ROS contains physical exam items”, “HPI missing critical symptom details”, “incorrect use of bullet points for this section”). In some implementations, revisions 338, 340, 342, 344 may be general or specific to a particular intermediary structured document such that revisions are made using intermediary structured document 306 for context.

In some implementations, automated prompt tuning process 10 generates 206 one or more revisions for the plurality of predefined prompts by modifying the original predefined prompts 304 to better accommodate the identified corrections from curated structured document 308. In one example, automated prompt tuning process 10 generates 206 one or more revisions in the form of modifying examples of the types of errors. In another example, automated prompt tuning process 10 generates 206 one or more revisions by editing the structure of text of predefined prompts 304. In another example, automated prompt tuning process 10 generates 206 one or more revisions by adding chain or tree of thought (i.e., directing generative AI model 300 to “reason” through a problem or task step by step, rather than jumping directly to a final answer or output). In this example and in the context of structured document generation, such as medical notes, chain of thought prompting can help ensure that each section of the document is logically derived from the preceding information. For instance, generative AI model 300 may be prompted to first summarize a patient's history, then describe the physical findings, and finally synthesize these into an assessment and plan, mirroring the clinician's reasoning process. In another example, automated prompt tuning process 10 generates 206 one or more revisions by ensuring self-consistency (i.e., by standardizing the structure, language, and expectations communicated to generative AI model 300). While several examples of different types of revisions to predefined prompts 304 have been provided, it will be appreciated that these are for example purposes only and that other prompt engineering approaches for revising predefined prompts are within the scope of the present disclosure.

In some implementations, generating 206 the one or more revisions for the plurality of predefined prompts includes generating 214 a plurality of revisions to be applied incrementally to the plurality of predefined prompts over a plurality of updates of the plurality of predefined prompts using a predefined relative prioritization. For example, certain sections may receive greater scores than other sections. Accordingly, automated prompt tuning process 10 may generate 206 revisions that are projected to have the most significant impact on the quality and accuracy of intermediary structured document 306 in light of these scores. However, the application of all revisions to a prompt for a particular intermediary structured document may result in revisions that are too specific for other common examples of the intermediary structured document. Further, the updating of predefined prompts may require significant computing resources. Accordingly, automated prompt tuning process 10 uses a predefined relative prioritization (e.g., predefined relative prioritization 346) to prioritize particular sections and revisions to sections. In some implementations, predefined relative prioritization 346 is a listing of particular sections to prioritize and/or particular revisions to prioritize over others. In one example, predefined relative prioritization 346 is user-defined. In another example, predefined relative prioritization 346 is defined or automatically updated by automated prompt tuning process 10. Using predefined relative prioritization 346, automated prompt tuning process 10 generates 214 a plurality of revisions in a schedule of revisions to be applied incrementally to the plurality of predefined prompts over a period of time and/or a sequence of updates to the plurality of predefined prompts. For example, automated prompt tuning process 10 may define a schedule of future updates to apply to the plurality of predefined prompts by prioritizing certain revisions for each future update.

In some implementations, automated prompt tuning process 10 updates the plurality of predefined prompts using the plurality of revisions. Referring again to FIG. 3, automated prompt tuning process 10 updates predefined prompts 302 with revisions 338, 340, 342, 344 to generate an updated plurality of predefined prompts (e.g., predefined prompts 302). In one example, automated prompt tuning process 10 incrementally applies the plurality of predefined prompts over a plurality of updates to gradually tune the prompts of first generative AI model 300. In this manner, the performance (i.e., accuracy and objectivity) of first generative AI model 300 can be monitored gradually as incremental revisions are applied to the plurality of predefined prompts (e.g., predefined prompts 302). For example, automated prompt tuning process 10 may generate a subsequent intermediary structured document using the updated plurality of predefined prompts and may compare the number and types of revisions generated for the subsequent intermediary structured document. In one example, if the number and/or type of revisions is reduced, automated prompt tuning process 10 may apply further revisions from the plurality of revisions to be applied incrementally. In another example, if the number and/or type of revisions is increased, automated prompt tuning process 10 may roll back the previous revision(s) to plurality of predefined prompts 302. In this manner, automated prompt tuning process 10 manages the updating of predefined prompts 302 to ensure that first generative AI model 300 generates increasingly accurate intermediary structured documents.

### General

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object-oriented programming language such as Java, Smalltalk, C++ or the like. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet (e.g., network 14).

The present disclosure is described with reference to flowchart illustrations and/or block diagrams of methods, apparatus (systems) and computer program products according to implementations of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general-purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods, and computer program products according to various implementations of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular implementations only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various implementations with various modifications as are suited to the particular use contemplated.

A number of implementations have been described. Having thus described the disclosure of the present application in detail and by reference to implementations thereof, it will be apparent that modifications and variations are possible without departing from the scope of the disclosure defined in the appended claims.

What is claimed is:

1. A computer-implemented method, executed on a computing device, comprising:
processing an intermediary structured document generated by a first generative artificial intelligence (AI) model using a plurality of predefined prompts;
comparing the intermediary structured document with a curated structured document;
generating a scoring of the intermediary structured document based upon, at least in part, the comparing of the intermediary structured document with a curated structured document; and
generating one or more revisions for the plurality of predefined prompts by processing the scoring of the intermediary structured document using a second generative AI model.

2. The computer-implemented method of claim 1, wherein the intermediary structured document is a medical record generated by the first generative AI model using the plurality of predefined prompts and medical data.

3. The computer-implemented method of claim 2, wherein the curated structured document is a medical record annotated by a medical professional.

4. The computer-implemented method of claim 3, wherein comparing the intermediary structured document with a curated structured document includes parsing the intermediary structured document into a plurality of sections and the curated structured document into a plurality of sections.

5. The computer-implemented method of claim 4, wherein comparing the intermediary structured document with a curated structured document includes comparing each section of the plurality of sections from the intermediary structured document with each corresponding section of the plurality of sections from the curated structured document.

6. The computer-implemented method of claim 5, wherein generating the scoring of the intermediary structured document includes generating a weighted score for each section of the plurality of sections from the intermediary structured document using a plurality of weights.

7. The computer-implemented method of claim 1, wherein generating the one or more revisions for the plurality of predefined prompts includes generating a plurality of revisions to be applied incrementally to the plurality of predefined prompts over a plurality of updates of the plurality of predefined prompts using a predefined relative prioritization.

8. A computing system comprising:
a memory; and
a processor to process an intermediary structured document generated by a first generative artificial intelligence (AI) model using a plurality of predefined prompts, to compare the intermediary structured document with a curated structured document, to generate a scoring of the intermediary structured document based upon, at least in part, the comparing of the intermediary structured document with a curated structured document, and to generate one or more revisions for the plurality of predefined prompts by processing the scoring of the intermediary structured document using a second generative AI model.

9. The computing system of claim 8, wherein the intermediary structured document is a medical record generated by the first generative AI model using the plurality of predefined prompts and medical data.

10. The computing system of claim 9, wherein the curated structured document is a medical record annotated by a medical professional.

11. The computing system of claim 10, wherein comparing the intermediary structured document with a curated structured document includes parsing the intermediary structured document into a plurality of sections and the curated structured document into a plurality of sections.

12. The computing system of claim 11, wherein comparing the intermediary structured document with a curated structured document includes comparing each section of the plurality of sections from the intermediary structured document with each corresponding section of the plurality of sections from the curated structured document.

13. The computing system of claim 12, wherein generating the scoring of the intermediary structured document includes generating a weighted score for each section of the plurality of sections from the intermediary structured document using a plurality of weights.

14. The computing system of claim 8, wherein generating the one or more revisions for the plurality of predefined prompts includes generating a plurality of revisions to be applied incrementally to the plurality of predefined prompts over a plurality of updates of the plurality of predefined prompts using a predefined relative prioritization.

15. A computer program product residing on a non-transitory computer readable medium having a plurality of instructions stored thereon which, when executed by a processor, cause the processor to perform operations comprising:
processing an intermediary structured document generated by a first generative artificial intelligence (AI) model using a plurality of predefined prompts;
comparing the intermediary structured document with a curated structured document;
generating a scoring of the intermediary structured document based upon, at least in part, the comparing of the intermediary structured document with a curated structured document; and
generating one or more revisions for the plurality of predefined prompts by processing the scoring of the intermediary structured document using a second generative AI model.

16. The computer program product of claim 15, wherein the intermediary structured document is a medical record generated by the first generative AI model using the plurality of predefined prompts and medical data.

17. The computer program product of claim 16, wherein the curated structured document is a medical record annotated by a medical professional.

18. The computer program product of claim 17, wherein comparing the intermediary structured document with a curated structured document includes parsing the intermediary structured document into a plurality of sections and the curated structured document into a plurality of sections.

19. The computer program product of claim 18, wherein comparing the intermediary structured document with a curated structured document includes comparing each section of the plurality of sections from the intermediary structured document with each corresponding section of the plurality of sections from the curated structured document.

20. The computer program product of claim 19, wherein generating the scoring of the intermediary structured document includes generating a weighted score for each section of the plurality of sections from the intermediary structured document using a plurality of weights.
